SEQUENCE LISTING (16 sequences)

SEQ ID NO: 1 - protein, 10 aa, Artificial Sequence
  Synthetic C-terminal epitope sequence of the C5 domain of the alpha-3 chain of type VI collagen against which the monoclonal antibody PRO-C6 is raised
KPGVISVMGT

SEQ ID NO: 2 - protein, 11 aa, Artificial Sequence
  Elongated peptide sequence
KPGVISVMGTA

SEQ ID NO: 3 - protein, 9 aa, Artificial Sequence
  Truncated peptide sequence
KPGVISVMG

SEQ ID NO: 4 - protein, 16 aa, Artificial Sequence
  Light chain complementary determining region CDR-L1 sequence
RSSQRIVHSNGITFLE

SEQ ID NO: 5 - protein, 7 aa, Artificial Sequence
  Light chain complementary determining region CDR-L2 sequence
RVSNRFS

SEQ ID NO: 6 - protein, 9 aa, Artificial Sequence
  Light chain complementary determining region CDR-L3 sequence
FQGSHVPLT

SEQ ID NO: 7 - protein, 5 aa, Artificial Sequence
  Heavy chain complementary determining region CDR-H1 sequence
DFNMN

SEQ ID NO: 8 - protein, 17 aa, Artificial Sequence
  Heavy chain complementary determining region CDR-H2 sequence
AINPHNGATSYNQKFSG

SEQ ID NO: 9 - protein, 7 aa, Artificial Sequence
  Heavy chain complementary determining region CDR-H3 sequence
WGNGKNS

SEQ ID NO: 10 - protein, 79 aa, Artificial Sequence
  Light chain complementary determining regions and framework sequence
RSSQRIVHSNGITFLEWYLQKPGQSPKLLIYRVSNRFSGVPDRFSGSGSGTDFTLKISRVEAEDLGLYYCFQGSHVPLT

SEQ ID NO: 11 - protein, 75 aa, Artificial Sequence
  Heavy chain complementary determining regions and framework sequence
DFNMNWVKQSHGKSLEWIGAINPHNGATSYNQKFSGKATLTVDKSSSTAYMELNSLTSDDSAVYYCARWGNGKNS

SEQ ID NO: 12 - protein, 112 aa, Artificial Sequence
  Light chain variable region sequence
DVVMTQTPLSLPVNLGDQASISCRSSQRIVHSNGITFLEWYLQKPGQSPKLLIYRVSNRFSGVPDRFSGSGSGTDFTLKISRVEAEDLGLYYCFQGSHVPLTFGAGTRLELK

SEQ ID NO: 13 - protein, 116 aa, Artificial Sequence
  Heavy chain variable region sequence
EVQLQQSGPVMVKPGTSVKTSCKASGYTFTDFNMNWVKQSHGKSLEWIGAINPHNGATSYNQKFSGKATLTVDKSSSTAYMELNSLTSDDSAVYYCARWGNGKNSWGQGTTLTVSS

SEQ ID NO: 14 - protein, 440 aa, Artificial Sequence
  Heavy chain sequence of monoclonal antibody PRO-C6
EVQLQQSGPVMVKPGTSVKTSCKASGYTFTDFNMNWVKQSHGKSLEWIGAINPHNGATSYNQKFSGKATLTVDKSSSTAYMELNSLTSDDSAVYYCARWGNGKNSWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 15 - protein, 219 aa, Artificial Sequence
  Heavy chain sequence of monoclonal antibody PRO-C6
DVVMTQTPLSLPVNLGDQASISCRSSQRIVHSNGITFLEWYLQKPGQSPKLLIYRVSNRFSGVPDRFSGSGSGTDFTLKISRVEAEDLGLYYCFQGSHVPLTFGAGTRLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - protein, 10 aa, Artificial Sequence
  Biotinylated PRO-C6 epitope sequence
  SITE 1: Biotin
KPGVISVMGT

(19) United States
(12) Patent Application Publication
(10) Pub. No.: US 2024/0125802 A1
(43) Pub. Date: Apr. 18, 2024

(54) Immunoassay for Detecting Eosinophilic Esophagitis

(71) Applicant: Nordic Bioscience A/S, Herlev (DE)

(72) Inventors: Joachim Høg Mortensen, Hundige Strand (DK); Martin Pehrsson, Skibby (DK); Morten Asser Karsdal, Kobenhavn Ø (DK)

(73) Assignee: Nordic Bioscience A/S, Herlev (DK)

(21) Appl. No.: 18/277,237

(22) PCT Filed: Feb. 17, 2022
(86) PCT No.: PCT/EP2022/053900

§ 371 Date: Aug. 15, 2023

(30) Foreign Application Priority Data
Feb. 18, 2021 (GB) 2102277.7

(51) Int. Cl. G01N 33/68; C07K 16/18

(52) U.S. Cl. CPC G01N 33/6893; C07K 16/18; C07K 2317/34; C07K 2317/565; G01N 2333/78; G01N 2800/14; G01N 2800/7052

(57) ABSTRACT

A method of immunoassay for detecting and/or monitoring esophageal fibrosis and/or dysphagia in a patient and/or assessing the likelihood of or the severity of esophageal fibrosis and/or dysphagia in a patient by contacting a biofluid sample from a patient with a monoclonal antibody that specifically binds to a C-terminal epitope of the C5 domain of the α3 chain of type VI collagen.

The present invention relates to detecting esophageal fibrosis and dysphagia by using an immunoassay to a marker of eosinophilic esophagitis, an epitope present at the C-terminus of the collagen type VI α3 chain.

Eosinophilic esophagitis (EoE) is an allergen/immune-mediated fibrostenotic disease of the esophagus. Progressive sub-epithelial fibrosis with subsequent risk for esophageal strictures over time may be present, which may result in dysphagia and food impaction, even with resolution of superficial mucosal eosinophilic inflammation.

Collagen Type VI is a unique extracellular collagen which can form an independent microfibrillar network in the basement membrane of cells. It can interact with other matrix proteins including collagens, biglycan, and proteoglycans. In muscle, type VI collagen is part of the sarcolemma and is involved in anchoring the muscle fiber into the intramuscular extracellular matrix, and so is involved in force transmission. Moreover, mutations in type VI collagen can cause Bethlem myopathy and Ullrich congenital muscular dystrophy. It has been reported that the C-terminal amino acid sequence of the type VI collagen α3 chain is cleaved off from the mature type VI microfibril after secretion. However, Type VI collagen is not just involved in muscles and muscle loss.

The microflamentous interstitial type VI collagen, a triple helical molecule composed of the constituent chains α1(VI), α2(VI), and α3(VI), is expressed in most connective tissues and prominently in adipose tissue, where it anchors cells through its interconnections with other ECM proteins. During the formation of microfilaments, the triple-helical core of type VI collagen is proteolytically released from the pro-peptide, and cleavage of the C-terminal pro-peptide of the α3(VI) chain generates endotrophin, an adipokine.

PRO-C6 is a biomarker for formation of collagen type VI and endotrophin release, comprising a C-terminal epitope of the C5 domain of the α3 chain of type VI collagen that is cleaved off when a novel collagen type VI molecule assembles in the extracellular matrix, and which C-terminal epitope is also a C-terminal epitope of the bioactive fragment endotrophin. The PRO-C6 biomarker, and a PRO-C6 assay (specifically, a PRO-C6 ELISA) are described in WO2016/156526. The assay utilizes a monoclonal antibody that specifically binds to the C-terminus 10 amino acid sequence of the C5 domain of the α3 chain of collagen type VI. Endotrophin's role as a pro-fibrotic, pro-inflammatory and pro-tumorigenic molecule has been observed in preclinical models of breast cancer and liver fibrosis (1-5). PRO-C6 has been established as a prognostic biomarker for mortality and disease progression in chronic kidney disease and diabetic kidney disease patients (6-8) and as a predictive marker for response to glucose lowering therapy in diabetic patients (9).

The present inventors have now identified PRO-C6 as a marker EoE to detect esophageal fibrosis and dysphagia.

Accordingly, in a first aspect the present invention provides a method of immunoassay for detecting and/or monitoring esophageal fibrosis and/or dysphagia in a patient and/or assessing the likelihood of or the severity of esophageal fibrosis and/or dysphagia in a patient, wherein said method comprises:
(i) contacting a biofluid sample from a patient with a monoclonal antibody that specifically binds to a C-terminal epitope of the C5 domain of the α3 chain of type VI collagen,
(ii) detecting and determining the amount of binding between the monoclonal antibody used in step (i) and peptides in the sample or samples, and
(iii) correlating said amount of binding of each monoclonal antibody as determined in step (ii) with values associated with normal healthy subjects and/or values associated with known disease severity and/or values obtained from said patient at a previous time point and/or a predetermined cut-off value.

The immunoassay may be, but is not limited to, a competition assay or a sandwich assay. The immunoassay may, for example, be a radioimmunoassay or an enzyme-linked immunosorbent assay (ELISA). Such assays are techniques known to the person skilled in the art.

The patient biofluid sample may be, but is not limited to, blood, serum, plasma, urine or amniotic fluid. Preferably the biofluid is serum or plasma.

As used herein the term “monoclonal antibody” refers to both whole antibodies and to fragments thereof that retain the binding specificity of the whole antibody, such as for example a Fab fragment, F(ab′)2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity.

In the methods of the present invention, a monoclonal antibody comprising any constant region known in the art can be used. Human constant light chains are classified as kappa and lambda light chains. Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgGI, IgG2, IgG3, and IgG4. The monoclonal antibody may preferably be of the IgG isotype, including any one of IgGI, IgG2, IgG3 or IgG4.

The CDR of an antibody can be determined using methods known in the art such as that described by Kabat et al. Antibodies can be generated from B cell clones as described in the examples. The isotype of the antibody can be determined by ELISA specific for human IgM, IgG or IgA isotype, or human IgG1, IgG2, IgG3 or IgG4 subclasses. The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

In some embodiments of the methods according to the first aspect of the invention, the biofluid sample is contacted with a monoclonal antibody which specifically binds to a C-terminal epitope of the C5 domain of the α3 chain of type VI collagen. Preferably said monoclonal antibody specifically binds to the C-terminus amino acid sequence KPGVISVMGT (SEQ ID No: 1) (also referred to herein as the “PRO-C6 sequence”, or simply “PRO-C6”). Preferably said monoclonal antibody does not recognize or specifically bind to an elongated version of said C-terminus amino acid sequence which is KPGVISVMGTA (SEQ ID No: 2), or to a truncated version of said C-terminus amino acid sequence which is KPGVISVMG (SEQ ID No: 3).

Preferably, the ratio of the affinity of said antibody for the C-terminus amino acid sequence KPGVISVMGT (SEQ ID No: 1) to the affinity of said antibody for the elongated C-terminus amino acid sequence KPGVISVMGTA (SEQ ID No: 2), and/or for the truncated C-terminus amino acid sequence KPGVISVMG (SEQ ID No: 3), is at least 10 to 1, and more preferably is at least 50 to 1, at least 100 to 1, at least 500 to 1, at least 1,000 to 1, at least 10,000 to 1, at least 100,000 to 1, or at least 1,000,000 to 1.

As used herein the term “C-terminus” refers to a C-terminal peptide sequence at the extremity of a polypeptide, i.e. at the C-terminal end of the polypeptide, and is not to be construed as meaning in the general direction thereof.

The monoclonal antibody that specifically binds to the PRO-C6 sequence may preferably comprises one or more complementarity-determining regions (CDRs) selected from:

CDR-L1
(SEQ ID No: 4)
RSSQRIVHSNGITFLE
CDR-L2:
(SEQ ID No: 5)
RVSNRFS
CDR-L3:
(SEQ ID No: 6)
FQGSHVPLT
CDR-H1:
(SEQ ID No: 7)
DFNMN
CDR-H2:
(SEQ ID No: 8)
AINPHNGATSYNQKFSG
CDR-H3:
(SEQ ID No: 9)
WGNGKNS.

Preferably the antibody comprises at least 2, 3, 4, 5 or 6 of the above listed CDR sequences.

Preferably the monoclonal antibody light chain variable region comprises the CDR sequences

CDR-L1:
(SEQ ID No: 4)
RSSQRIVHSNGITFLE
CDR-L2:
(SEQ ID No: 5)
RVSNRFS
and
CDR-L3:
(SEQ ID No: 6)
FQGSHVPLT.

Preferably the monoclonal antibody light chain comprises framework sequences between the CDRs, wherein said framework sequences are substantially identical or substantially similar to the framework sequences between the CDRs in the light chain sequence below (in which the CDRs are shown in bold and underlined, and the framework sequences are shown in italics)

(SEQ ID NO: 10)
RSSQRIVHSNGITFLEWYLQKPGQSPKLLIYRVSNRFSGVPDRFSGSGS
GTDFTLKISRVEAEDLGLYYCFQGSHVPLT.

Preferably the monoclonal antibody heavy chain variable region comprises the CDR sequences

CDR-H1:
(SEQ ID No: 7)
DFNMN
CDR-H2:
(SEQ ID No: 8)
AINPHNGATSYNQKFSG
CDR-H3:
(SEQ ID No: 9)
WGNGKNS.

Preferably the monoclonal antibody heavy chain comprises framework sequences between the CDRs, wherein said framework sequences are substantially identical or substantially similar to the framework sequences between the CDRs in the heavy chain sequence below (in which the CDRs are shown in bold and underlined, and the framework sequences are shown in italics)

(SEQ ID NO: 11)
DFNMNWVKQSHGKSLEWIGAINPHNGATSYNQKFSGKATLTVDKSSSTA
YMELNSLTSDDSAVYYCARWGNGKNS.

As used herein, the framework amino acid sequences between the CDRs of an antibody are substantially identical or substantially similar to the framework amino acid sequences between the CDRs of another antibody if they have at least 70%, 80%, 90% or at least 95% similarity or identity. The similar or identical amino acids may be contiguous or non-contiguous.

The framework sequences may contain one or more amino acid substitutions, insertions and/or deletions. Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 Asp, Asn, Glu, Gln; Group 3 His, Arg, Lys; Group 4 Met, Leu, Ile, Val, Cys; Group 5 Phe Thy Trp.

A program such as the CLUSTAL program to can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention. Identity or similarity is preferably calculated over the entire length of the framework sequences.

In certain preferred embodiments, the monoclonal antibody that specifically binds to the PRO-C6 sequence may comprise the light chain variable region sequence:

(SEQ ID No: 12)
DVVMTQTPLSLPVNLGDQASISCRSSQRIVHSNGITFLEWYLQKPGQSP
KLLIYRVSNRFSGVPDRFSGSGSGTDFTLKISRVEAEDLGLYYCFQGSH
VPLTFGAGTRLELK
and/or
the heavy chain variable region sequence:
(SEQ ID No: 13)
EVQLQQSGPVMVKPGTSVKTSCKASGYTFTDFNMNWVKQSHGKSLEWIG
AINPHNGATSYNQKFSGKATLTVDKSSSTAYMELNSLTSDDSAVYYCAR
WGNGKNSWGQGTTLTVSS
(CDRs bold and underlined; Framework sequences in italics)

In some embodiments of the methods according to the first aspect of the invention, the amount of binding of the monoclonal antibody specific for the C-terminal epitope of the C5 domain of the α3 chain of collagen type VI, are correlated with values associated with normal healthy subjects and/or with values associated with known disease severity and/or with values obtained from the patient at a previous point in time.

As used herein the term “values associated with normal healthy subjects and/or values associated with known disease severity” means standardised quantities determined by the method described supra for subjects considered to be healthy, i.e. without a cardiovascular disease, and/or standardised quantities determined by the method described supra for subjects known to have a EoE with a known severity.

In some embodiments of the method according to the first aspect, the amount of binding of the monoclonal antibody specific for the C-terminal epitope of the C5 domain of the α3 chain of collagen type VI are compared with one or more predetermined cut-off values.

As used herein the “cut-off value” means an amount of binding that is determined statistically to be indicative of a high likelihood of EoE in a patient, or of EoE of a particular level of severity, in that a measured value of biomarker binding in a patient sample that is at or above the statistical cutoff value corresponds to at least a 70% probability, preferably at least an 80% probability, preferably at least an 85% probability, more preferably at least a 90% probability, and most preferably at least a 95% probability of the presence or likelihood of EoE or of a particular level of severity of the disease.

The predetermined cut-off value for the amount of binding of the monoclonal antibody specific for the C-terminal epitope of the C5 domain of the α3 chain of collagen type VI is preferably at least 9.0 ng/mL, more preferably at least 12.0 ng/mL. By having a statistical cut-off value of at least 9.0 ng/mL, and more preferably at least 12.0 ng/mL it is possible to utilise the method of the invention to give a prognosis of EoE with a high level of confidence. Applying such statistical cut-off values are particularly advantageous as it results in a standalone diagnostic assay; i.e. it removes the need for any direct comparisons with healthy individuals and/or patients with known disease severity in order to arrive at a diagnostic conclusion. This may also be particularly advantageous when utilising the assay to evaluate patients that already have medical signs or symptoms that are generally indicative of EoE (e.g. as determined by a physical examination and/or consultation with a medical professional) as it may act as a quick and definitive tool for corroborating the initial prognosis and thus potentially remove the need for more invasive procedures, and expedite the commencement of a suitable treatment regimen. It may also avoid the need for a lengthy hospital stay.

### FIGURES

FIG. 1 shows PRO-C6 serum levels in EoE patients stratified according to (a) the Schatizki rings/fibrostenosis and (b) EREF fibrosis score.

FIG. 2 shows PRO-C6 serum levels in EoE patients stratified according to EoE patients and dysphagia.

### EXAMPLES

### Example 1—Antibody Development for Pro-C6

A monoclonal antibody specific for Pro-C6 was developed as described in WO 2016/156526 (Nordic Bioscience, incorporated herein by reference) using the last 10 amino acids of the type VI collagen α3 chain (i.e. the C-terminus sequence ³¹⁶⁸‘KPGVISVMGT’³¹⁷⁷ (SEQ ID No: 1)) as an immunogenic peptide. Briefly, 4-6-week-old Balb/C mice were immunized subcutaneously with 200 μl emulsified antigen with 60 μg of the immunogenic peptide. Consecutive immunizations were performed at 2-week intervals in Freund's incomplete adjuvant, until stable sera titer levels were reached, and the mice were bled from the 2nd immunization on. At each bleeding, the serum titer was detected and the mouse with highest antiserum titer and the best native reactivity was selected for fusion. The selected mouse was rested for 1 month followed by intravenous boosting with 50 μg of immunogenic peptide in 100 μl 0.9% sodium chloride solution 3 days before isolation of the spleen for cell fusion.

Mouse spleen cells were fused with SP2/0 myeloma fusion partner cells. The fusion cells were raised in 96-well plates and incubated in the CO₂-incubator. Here standard limited dilution was used to promote monoclonal growth. Cell lines specific to the selection peptide and without cross-reactivity to either elongated peptide (KPGVISVMGTA (SEQ ID No: 2), Chinese Peptide Company, China) or truncated peptide (KPGVISVMG (SEQ ID No: 3), American Peptide Company, USA) were selected and sub-cloned. At last the antibodies were purified using an IgG column.

The antibodies generated were sequenced and the CDRs determined.

The sequence of the chains are as follows (CDRs underlined and in bold):

### Heavy Chain Sequence (Mouse IgG1 Isotype)

(SEQ ID No: 14)
EVQLQQSGPVMVKPGTSVKTSCKASGYTFTDFNMNWVKQSHGKSLEWIG
AINPHNGATSYNQKFSGKATLTVDKSSSTAYMELNSLTSDDSAVYYCAR
WGNGKNSWGQGTTLTVSSAKTTPPSVYPLAPGSAAQTNSMVTLGCLVKG
YFPEPVTVTWNSGSLSSGVHTFPAVLQSDLYTLSSSVTVPSSTWPSETV
TCNVAHPASSTKVDKKIVPRDCGCKPCICTVPEVSSVFIFPPKPKDVLT
ITLTPKVTCVVVDISKDDPEVQFSWFVDDVEVHTAQTQPREEQFNSTFR
SVSELPIMHQDWLNGKEFKCRVNSAAFPAPIEKTISKTKGRPKAPQVYT
IPPPKEQMAKDKVSLTCMITDFFPEDITVEWQWNGQPAENYKNTQPIMD
TDGSYFVYSKLNVQKSNWEAGNTFTCSVLHEGLHNHHTEKSLSHSPGK
CDR-H1:
(SEQ ID No: 7)
DFNMN
CDR-H2:
(SEQ ID No: 8)
AINPHNGATSYNQKFSG
CDR-H3:
(SEQ ID No: 9)
WGNGKNS
Light Chain Sequence (mouse Kappa isotype)
(SEQ ID No: 15)
DVVMTQTPLSLPVNLGDQASISCRSSQRIVHSNGITFLEWYLQKPGQSP
KLLIYRVSNRFSGVPDRFSGSGSGTDFTLKISRVEAEDLGLYYCFQGSH
VPLTFGAGTRLELKRADAAPTVSIFPPSSEQLTSGGASVVCFLNNFYPK
DINVKWKIDGSERQNGVLNSWTDQDSKDSTYSMSSTLTLTKDEYERHNS
YTCEATHKTSTSPIVKSFNRNEC
CDR-L1:
(SEQ ID No: 4)
RSSQRIVHSNGITFLE
CDR-L2:
(SEQ ID No: 5)
RVSNRFS
CDR-L3:
(SEQ ID No: 6)
FQGSHVPLT

### Example 2. PRO-C6 Immunoassay

PRO-C6 was measured using an enzyme-linked immunosorbent assay (ELISA) developed at Nordic Bioscience, as described in WO2016/156526, and as also detailed in other publications. Briefly, these procedures were as follows:

ELISA-plates used for the assay development were Streptavidin-coated from Roche (cat.: 11940279). All ELISA plates were analyzed with the ELISA reader from Molecular Devices, SpectraMax M, (CA, USA). We labeled the selected monoclonal antibody with horseradish peroxidase (HRP) using the Lightning link HRP labeling kit according to the instructions of the manufacturer (Innovabioscience, Babraham, Cambridge, UK). A 96-well streptavidin plate was coated with biotinylated synthetic peptide biotin-KPGVISVMGT (SEQ ID No: 16) (Chinese Peptide Company, China) dissolved in coating buffer (40 mM Na₂HPO₄, 7 mM KH₂PO₄, 137 mM NaCl, 2.7 mM KCl, 0.1% Tween 20, 1% BSA, pH 7.4) and incubated 30 minutes at 20° C. 20 μL of standard peptide or samples diluted in incubation buffer (40 mM Na₂HPO₄, 7 mM KH₂PO₄, 137 mM NaCl, 2.7 mM KCl, 0.1% Tween 20, 1% BSA, 5% Liquid II, pH 7.4) were added to appropriate wells, followed by 100 μL of HRP conjugated monoclonal antibody 10A3, and incubated 21 hour at 4° C. Finally, 100 μL tetramethylbenzinidine (TMB) (Kem-En-Tec cat.438OH) was added and the plate was incubated 15 minutes at 20° C. in the dark. All the above incubation steps included shaking at 300 rpm. After each incubation step the plate was washed five times in washing buffer (20 mM Tris, 50 mM NaCl). The TMB reaction was stopped by adding 100 μL of stopping solution (1% H₂SO₄) and measured at 450 nm with 650 nm as the reference.

### Example 3

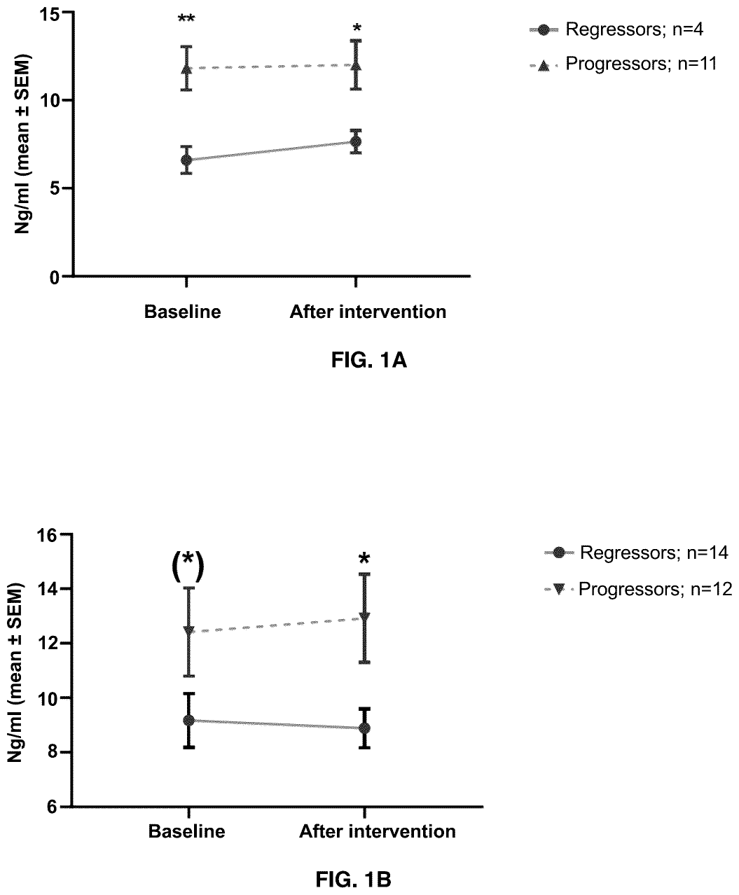

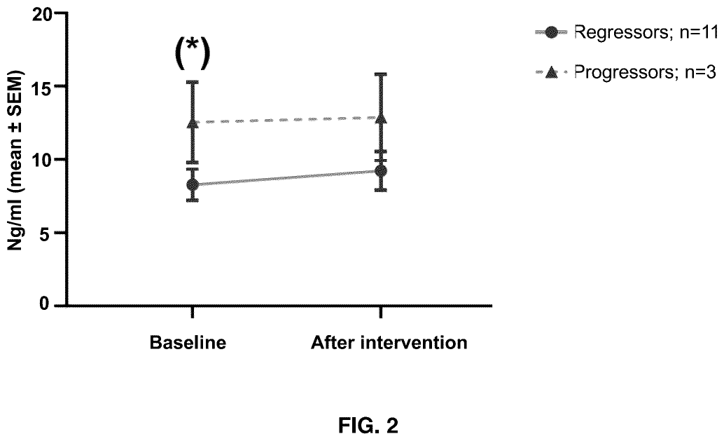

Serum samples from 30 adult EoE patients on an elimination diet (60% males, median age 36.5 years, median disease duration 8.0 years,) were included for analysis at baseline and 30 days after intervention. No patients were diagnosed with co-morbidities at diagnosis or time of sampling. Serum levels of PRO-C6 were assessed in the EoE patients and age/gender matched healthy donors (n=−30). Schatzki rings, fibrostenosis and EREF subscore for fibrosis were used to evaluate the presence of fibrosis at baseline and after intervention. In addition, dysphagia was also evaluated in the EoE patients. The EoE patients were stratified as regressors (decrease in fibrosis score from baseline to after intervention) or progressors (increase in fibrosis score from baseline to after intervention) of fibrosis for Schatzki rings/fibrostenosis (regressors: n=4, progressors: n=11) and EREF fibrosis (regressors: n=14, progressors: n=12). EoE patients had significantly elevated PRO-C6 serum levels compared to healthy donors. In addition, we observed significantly elevated serum levels type VI collagen formation PRO-C6 in patients presenting with a progressive fibrotic phenotype at both timepoints (FIG. 1). In addition, patients without dysphagia (n=11) also showed numerical lower levels of PRO-C6 compared to patients with dysphagia (n=3) at baseline and after intervention (FIG. 2).

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

1. Park J, Scherer P E, Calle E et al. Adipocyte-derived endotrophin promotes malignant tumor progression. J. Clin. Invest. 2012; 122: 4243-4256.
2. Lee C, Kim M, Lee J H et al. COL6A3-derived endotrophin links reciprocal interactions among hepatic cells in the pathology of chronic liver disease. J. Pathol. 2018.
3. Sun K, Park J, Gupta O T et al. Endotrophin triggers adipose tissue fibrosis and metabolic dysfunction. Nat. Commun. 2014; 5: 3485.
4. Park J, Morley T S, Scherer P E. Inhibition of endotrophin, a cleavage product of collagen VI, confers cisplatin sensitivity to tumours. EMBO Mol. Med. 2013; 5: 935-48.
5. Bu D, Crewe C, Kusminski CM et al. Human endotrophin as a driver of malignant tumor growth. JCI Insight 2019.
6. Rasmussen D G K, Hansen T W, von Scholten B J et al. Higher Collagen VI Formation Is Associated With All-Cause Mortality in Patients With Type 2 Diabetes and Microalbuminuria. Diabetes Care 2018: dc172392.
7. Fenton A, Jesky M D, Ferro C J et al. Serum endotrophin, a type VI collagen cleavage product, is associated with increased mortality in chronic kidney disease. Aguilera A I, ed. PLoS One 2017; 12: e0175200.
8. Rasmussen D G K, Fenton A, Jesky M et al. Urinary endotrophin predicts disease progression in patients with chronic kidney disease. Sci. Rep. 2017; 7: 17328.
9. Karsdal M A, Henriksen K, Genovese F et al. Serum endotrophin identifies optimal responders to PPARγ agonists in type 2 diabetes. Diabetologia 2017; 60.

1. A method of immunoassay for detecting and/or monitoring esophageal fibrosis and/or dysphagia in a patient and/or assessing the likelihood of or the severity of esophageal fibrosis and/or dysphagia in a patient, wherein said method comprises:
(i) contacting a biofluid sample from a patient with a monoclonal antibody that specifically binds to a C-terminal epitope of the C5 domain of the α3 chain of type VI collagen,
(ii) detecting and determining the amount of binding between the monoclonal antibody used in step (i) and peptides in the sample or samples, and
(iii) correlating said amount of binding of each monoclonal antibody as determined in step (ii) with values associated with normal healthy subjects and/or values associated with known disease severity and/or values obtained from said patient at a previous time point and/or a predetermined cut-off value.

2. The method of claim 1, wherein said monoclonal antibody specifically binds to a C-terminus amino acid sequence KPGVISVMGT (SEQ ID NO: 1).

3. The method of claim 2, wherein said monoclonal antibody does not recognize or specifically bind to an elongated version of said C-terminus amino acid sequence which is KPGVISVMGTA (SEQ ID NO: 2), or to a truncated version of said C-terminus amino acid sequence which is KPGVISVMG (SEQ ID NO: 3).

4. The method of claim 1, wherein said biofluid is serum or plasma.

5. The method of claim 1, wherein said immunoassay is a competition assay or a sandwich assay.

6. The method of claim 1, wherein said immunoassay is a radioimmunoassay or an enzyme-linked immunosorbent assay.
